(19) United States
(12) Patent Application Publication
(10) Pub. No.: US 2004/0242514 A1
(43) Pub. Date: Dec. 2, 2004

(54) Xenogenic oligo or/and polyribonucleotides as agents for the treatment of malignant tumours

(76) Inventor: Barbara Meisriemler, Gotthard (AT)

Correspondence Address: FULBRIGHT & JAWORSKI, LLP, 666 FIFTH AVE, NEW YORK, NY, 10103-3198, US

(21) Appl. No.: 10/481,390

(22) PCT Filed: Jun. 26, 2002
(86) PCT No.: PCT/EP02/07071

(30) Foreign Application Priority Data
Jun. 28, 2001 (DE) 101 31 148.6

(51) Int. Cl. A61K 48/00

(52) U.S. Cl. 514/44

(57) ABSTRACT

The invention relates to xenogenic oligo- or/and polyribonucleotides as agents for the treatment of malignant tumors. The invention also relates to the use of said xenogenic oligo- or/and polyribonucleotides in the production of medicaments for the treatment of malignant tumors.

The invention relates to xenogenic oligo- or/and polyribonucleotides as agents for the treatment of malignant tumors. The invention also relates to the use of said xenogenic oligo- or/and polyribonucleotides in the production of medicaments for the treatment of malignant tumors.

### BACKGROUND OF THE INVENTION

In the late 1960s and early 1970s, it was found in the context of transplant research that tissue pretreated with xenogenic heterogeneous nucleic acids or weak antigens had substantially increased antititers in various immunological test methods. These results were confirmed further using a number of various antigens in in vitro and in vivo studies. However, there was no indication that nucleic acids and in particular oligo- or/and polyribonucleotides of xenogenic origin could be suitable for the treatment of malignant tumors.

At the same time, especially in the USA, experiments with defined synthetic poly- and oligonucleotides, particularly ribonucleotides, were carried out, which, however, were not pursued any further, due to the high toxicity in vivo.

It was therefore the object of the present invention to produce a medicament which is suitable for the treatment of malignant tumors. Malignant tumors in accordance with the present invention do not comprise malignant skin disorders.

According to the invention, the object is achieved by a method for producing a medicament for the treatment of malignant tumors, which medicament comprises as active compound xenogenic oligo- or/and polyribonucleotides, preferably in the form of their conjugates with cells of the tumor to be treated.

Xenogenic in accordance with the present invention means that the ribonucleic acid originates from an organism different from the one to be treated therewith, i.e. those oligo- or/and polyribonucleotides which are not from the same organism as that to which the medicament is to be administered. The xenogenic oligo- or/and polyribonucleotides used according to the invention are preferably those from animal tissues (e.g. bovine tissue, fetal calf tissue), plants and unicellular organisms, preferably from yeast cells (in particular Saccharomyces cerevisiae). Preference is given to using oligo- or/and polyribonucleotides of organisms which are evolutionary as distant as possible from the organism to be treated. Thus, preference is given to using in medicaments for humans RNA from animal tissues or, particularly preferably, from plants or unicellular organisms such as, for example, yeast.

The oligo- or/and polyribonucleotides used according to the invention are nontoxic and, on their own, nonantigenic.

It is possible to effectively use preparations of total RNA and salts and compounds thereof. Particular preference is given to tRNA. A particularly preferred manner of obtaining RNAs which can be used according to the invention is phenol extraction, specifically the methods referred to herein as methods I and II.

It was furthermore found that the xenogenic ribonucleic acids (RNA) in combination with peptides, polypeptides and proteins increase the antigenicity of the latter. Owing to these observations, tumor tissue of the affected patients was treated with xenogenic RNA both in vitro and in vivo. Preference is given to treating the cells of the tumor tissue of a patient with the xenogenic RNA, preferably with tRNA, in vitro. The mixture is then systemically administered to said patient. In the case of superficial malignant tumors, the RNA may also be applied topically in a suitable pharmaceutical form.

In addition to treating humans with the xenogenic oligo- or/and polyribonucleotides of the present invention, it is also possible to treat warmblooded animals such as, for example, horses, cattle, sheep, etc. in this way.

The following examples and experimental results further illustrate the invention.

### EXAMPLES

### Example 1

Production of the Oligo- or/and Polyribonucleotides Usable According to the Invention

The relevant literature describes a large number of methods for obtaining nucleic acids, nucleotides and nucleosides, which are known to anyone having the relevant experience. Two methods with small modifications, which are both based on phenolization, are preferably applied here, method I for obtaining the total RNA (Georgiev, G. P. and Mantieva, V. L., Biochim. Biophys. acta 61, 153 (1962)) and method II for obtaining the tRNA (Bauer, S. et al., Biotechnology and Bioengineering 15, 1081 (1973)). Both methods are suitable for extracting relatively large amounts.

Method I

A 15% suspension of brewer's yeast (Saccharomyces cerevisiae) was in buffer (A) [0.001 M EDTA, 0.01 M Tris-HCl buffer, pH 5-6, 25% sucrose, 0.5% SDS (sodium dodecyl sulfate), 0.3% Na deoxycholate] was homogenized in a Waring Blendor [sic] at 10° C. and 3000 rpm for 3 minutes. The homogenate was admixed with the same volume of solution (B) [80% recrystallized phenol in buffer (A); 0.1% 8-hydroxyquinoline, 1.2% diethyl pyrocarbonate] and then slowly stirred at 60° C. for 30 minutes. All buffer solutions were prepared with deionized water which had been agitated with bentonite beforehand. The phenolized homogenate was then centrifuged at room temperature, approx. 20° C., with 10000 g for 15 minutes. The aqueous phase was removed and the phenol and the intermediate phase were discarded. The aqueous phase was admixed with the same volume of a 1:1 mixture of solution (B) and chloroform/isoamyl alcohol (96:4) and extracted as described above. The aqueous phase was extracted three times with half the volume of diethyl ether in order to remove the remaining phenol. The solution was adjusted to 2% sodium acetate and the RNA was precipitated with 2.5 volumes of absolute ethanol.

The precipitated RNA was removed by centrifugation at 0° C. and 5000 rpm and taken up in an ice-cold 0.01 M Tris-HCl buffer, pH 7.0 and 0.001 M MgCl₂. Possible DNA was degraded by adding electrophoretically pure pancreatic DNase (4 μg/ml) to the solution and incubating at 22° C. for 3 hours. Protein residues, the DNase and RNases were digested with pronase (10 μg/ml) at 37° C. for 3 hours. During this time, pronase was also destroyed by digesting itself. The RNA solution was extracted as described above with solution (B) at 60° C. with gentle stirring for 20 minutes, the phases were separated by centrifugation, the aqueous phase was removed and extracted with diethyl ether. After addition of sodium acetate (final concentration 2%), the RNA was precipitated with 2.5 volumes of ethanol and removed by centrifugation. The precipitate was taken up in cold 2% strength sodium acetate, precipitated with 2.5 volumes of ethyl alcohol and left in the alcohol mixture at −20° C. overnight. The precipitate was then removed by centrifugation, and washed twice with 75% strength ethanol, twice with absolute ethanol and twice with diethyl ether. After drying in an oven, a loose-packed dry RNA was obtained, which was stored in a dark glass vessel at room temperature.

Method II

This method is also suitable for extracting large quantities of yeast (kilogram quantities).

A given weight [sic] of yeast was homogenized in four times the amount of buffer (A) (see method I above) in the cold room. 40% v/v of phenol solution (B) and 5% w/v ice cubes made of deionized water were added to the homogenate and the mixture was stirred for 30 minutes. The supernatant was removed by suction and then phenolized two more times, as described under method I. The aqueous supernatants were collected in a vessel which contained a DEAE-cellulose suspension (approx. 10% w/v, Whatman DE-22), corresponding to half the volume of the collected supernatants. The DEAE suspension was kept in suspension by stirring for 30 minutes. The DEAE was then allowed to sediment over one hour. The supernatant was removed by suction. In the meantime, the intermediate phase and phenol phase were stirred two more times with the aliquot amount of solution (C) (83% deionized water, 15% w/v ice cubes, 2% Mg-acetate concentrate [0.5M Mg-acetate in 0.25 [lacuna] mercaptoethanol] for 30 minutes and then allowed to separate for 70-80 minutes. The aqueous supernatants were transferred into the vessel containing DEAE, and then again stirred and allowed to sediment. The supernatant was removed by suction and the DEAE was washed, as above, first twice with solution C, then again with solution (D) (˜2 volumes of Mg-acetate concentrate, 2 volumes of NaCl concentrate [3.75 M NaCl in water], 0.2 volumes of Tris-HCl concentrate [2.5 M Tris-HCl, pH 7.5 in water, 96 volumes of water]).

DEAE-cellulose was then packed into a column which was closed at the bottom. All further steps were carried out in the cold room at 4° C. The column was washed with 12 times the amount of the column contents of solution (D), flow rate 1.4 l/h, (only by gravity). The tRNA was then eluted with solution E [2 volumes of Mg-acetate concentrate, 0.2 volumes of Tris-HCl concentrate, 14 volumes of NaCl concentrate and 84 volumes of water, final NaCl concentration 0.525 M, with a flow of 3 l/h. The fractions which contained more than 35 A₂₆₀ units/ml were combined and precipitated with 1.5 volumes of ethanol. The further procedure was according to method I.

Alternatively, the final precipitate can be taken up in water and can be lyophilized.

A variant of this method is the common phenolization of the starting material: crude tRNA is precipitated out of the upper phase with isopropanol. After centrifugation, the precipitate is extracted with the sodium acetate buffer and chromatographed on DEAE-cellulose. Elution is carried out with the sodium acetate/sodium chloride gradient, as it is known to biochemists experienced in the matter. The suitable fractions, see above, are determined by means of quotient measurement and combined. The tRNA is precipitated with ethanol, the precipitate is taken up as above and is preferably lyophilized.

The following assays were employed for analyzing the purity of the total RNA and tRNA and for characterizing them:

Protein was determined according to Lowry, O. H. et al. (J. Biol. Chem. 193, 265 (1951)) and by A₂₆₀/A₂₈₀≅2, DNA according to Dische (Mikrochemie 8, 4 (1930), total RNA according to Mejbaum (Physiol. Chem. 258, 117 (1939)), quantitative determination of tRNA and of amino acid incorporation according to Sprinzl and Sternbach (Methods in Enzymology 59, 182 (1979)) toxicity according to M. Nöldner (personal communication), absence of pyrogen in vitro according to DAB 1997 (LAL assay) and in vivo according to Ph. Eur./DAB 1997.

Results of the Analyses:

(Properties of total RNA and tRNA, averages from ten tests)

Absorption

A₂₆₀/A₂₈₀≅1.94-2.0

C, H, N analysis

| | | |
|---|---|---|
| C | 32.67 | 32.42 |
| H | 5.22 | 5.20 |
| N | 2.29 | 2.00 |

with corresponding values of various total RNAs and tRNAs.

UV and IR Spectra

The UV and IR spectra vary, they are almost the same but not identical, corresponding to biological substances.

Molecular Weight

Total RNA and tRNA from yeast±22000-27000 dalton average, varying for different preparations;

| Protein | DNA | (Total contents) |
|---|---|---|
| 2.3% | neg. | Total RNA of Saccharomyces cerevisiae |
| 1.9% | neg. | tRNA of Saccharomyces cerevisiae |
| 0.9% | neg. | Total RNA of bovine origin |

Average, generally common quality. Improved purity led to no significantly improved therapeutic action, at a disproportionally higher cost.

Amino acid incorporation for tRNA, average of 10 analyses

| | | |
|---|---|---|
| Lysine | 69-85 | pmol/A₂₆₀ unit |
| Phe | 41-55 | |
| Ser | 39-50 | |
| Val | 77-90 | |

These averages vary in yeasts of different lots within the range stated.

Toxicity

Test for acute toxicity in mice:

Animals: NMRI mice, male, Janvier, France

Administration: Intravenously into a tail vein

Observation period: 24 hours

Number of random samples: n=10 at highest concentration

Assay substance: a. bovine total RNA

b. tRNA from brewer's yeast (Saccharomyces cerevisiae)

Solvent: 0.9% NaCl in water p.i.

Result:

Up to a maximum dosage of 1 g/kg/10 ml i.v., the animals used in the test showed no conspicuous features whatsoever within the observation period of 24 hours.

Absence of Pyrogen

A. The pyrogen content of total RNA and tRNA, both as described previously, was determined using the in-vitro assay for endotoxins according to DAB 1997 (LAL TEST) and on rabbits according to Ph. Eur./DAB 1997.

1. Total RNA

Endotoxin standard EC 5

Amoebocyte lysate

Sensitivity declared: 0.06 EU/ml

Sensitivity found: 0.06 EU/ml

Test solution: 100 mg RNA dissolved in 20 ml of water-LAL (0.5%)

Result:

Endotoxin content of the test solution 0.5% 1:5 diluted with water-LAL: <0.03 EU/ml.

2. tRNA

Endotoxin standard EC 5

Amoebocyte lysate

Sensitivity declared: 0.06 EU/ml

Sensitivity found: 0.06 EU/ml

Test solution: 100 mg RNA dissolved in 20 ml of water-LAL (0.5%)

Result:

Endotoxin content of the test solution 0.5% 1:10 diluted with water-LAL: <0.03 EU/ml.

B. In Vivo Test for Absence of Pyrogen According to DAB/Ph.Eub., as of 2000

1. Total RNA

Test solution 1% of assay substance in pyrogen-free water p.i.

Dose: 1.0 ml/animal

Animals: 3 rabbits, corresponding to DAB/Ph. Eub., as of 2000

Result:

Sum of temperature differences of 3 rabbits was 1.05° C., thus pyrogens are not detectable.

2. tRNA

Test solution 1% of assay substance in pyrogen-free water p.i.

Dose: 1.0 ml/animal

Animals: 2 times 6 rabbits, corresponding to DAB/Ph. Eub., as of 2000

Result:

a. Sum of temperature differences of 6 rabbits: 1.02° C.

b. Sum of temperature differences of 6 rabbits: 1.06° C., pyrogens not detectable.

### Example 2

Detection of the Tumor Efficacy of the Substances of the Present Invention

10 patients who suffered from various carcinomas, had had surgery, whose bodies had been invaded by numerous metastases and who no longer responded to chemotherapy, whose life was estimated by the oncologists treating them to last a few weeks, were treated systemically with a tRNA/tumor cell conjugate, incubation approx. 30 minutes, 6×10⁶ tumor cells with 50-100 mg of tRNA, 2 times in 14 days.

After this treatment, a female patient was also treated supportively with a mild chemotherapeutic over a few weeks. After nearly 2 years, she died independently of her basic disorder.

All of the remaining patients treated survived >1-2 years without chemotherapy and with good quality of life without side effects.

These results justify the use of said RNA in patients in particular, since they no longer responded to chemotherapy were infaust, and no side effects or toxic effects whatsoever were observed during the prolonged life.

1-15. (canceled).

16. A method for producing a composition for the treatment of malignant tumors, with the proviso that said malignant tumor is not a skin tumor, comprising converting xenogenic oligo- or poly-ribonucleotides into a systemically administerable form:

17. The method of claim 16, wherein said xenogenic oligo- or poly-ribonucleotides are conjugated with cells of the malignant tumor to be treated.

18. The method of claim 17, wherein said conjugates are formed by incubating said xenogenic oligo- or poly-ribonucleotides in vitro with cells of the malignant tumor to be treated.

19. The method of claim 16, wherein said xenogenic oligo- or poly-ribonucleotides are combined with a physiologically acceptable carrier, excipient, diluent or additive.

20. The method of claim 16, wherein said oligo- or poly-ribonucleotides are obtained from an animal tissue, a plant or a unicellular organism.

21. The method of claim 20, wherein said oligo- or poly-ribonucleotides are obtained from yeast cells.

22. The method of claim 16, wherein said xenogenic oligo- or poly-ribonucleotides comprise xenogenic tRNA.

23. The method of claim 16, wherein said xenogenic oligo- or poly-ribonucleotides are obtained by phenol extraction of a sample which contains xenogenic oligo- or polyribonucleotides.

24. The method of claim 16, wherein said xenogenic oligo- or poly-ribonucleotides are derived from an organism which is evolutionarily distinct from a subject to be treated.

25. A method of treating a malignant tumor comprising administering a therapeutically effective amount of xenogenic oligo- or poly-ribonucleotides to a subject in need thereof, wherein said malignant tumor is not a skin tumor.

26. The method of claim 25, wherein said xenogenic oligo- and poly-ribonucleotides are conjugated with cells of the malignant tumor to be treated.

27. The method of claim 26, wherein said conjugates are formed by incubating said xenogenic oligo- and poly-ribonucleotides in vitro with cells of the malignant tumor to be treated.

28. The method of claim 25, wherein said xenogenic oligo- or poly-ribonucleotides comprise xenogenic tRNA.

29. The method of claim 28, comprising systemically administering said xenogenic oligo- and polyribonucleotides, in an active amount ranging from 50 mg to 100 mg of tRNA.

30. The method of claim 29, wherein said tRNA is conjugated with tumor cells of the malignant tumor to be treated.

31. The method of claim 30, wherein said conjugate is formed by incubating 105-108 tumor cells with 50-100 mg of tRNA in vitro, wherein said conjugate is systemically administered more than once to a subject in need thereof.
